(19) United States
(12) Patent Application Publication
(10) Pub. No.: US 2025/0160665 A1
(43) Pub. Date: May 22, 2025

(54) SELECTABLE HEART SOUND TRACKING IN HEART FAILURE

(71) Applicant: Cardiac Pacemakers, Inc., St. Paul, MN (US)

(72) Inventors: Mojgan Goftari, Shoreview, MN (US); Viktoria A. Averina, Shoreview, MN (US); Jonathan Bennett Shute, Eagan, MN (US)

(21) Appl. No.: 18/938,967

(22) Filed: Nov. 6, 2024

Related U.S. Application Data
(60) Provisional application No. 63/599,867, filed on Nov. 16, 2023.

(51) Int. Cl. A61B 5/024; A61B 5/00

(52) U.S. Cl. CPC A61B 5/024; A61B 5/0075; A61B 5/7282

(57) ABSTRACT

Systems and methods for recognizing and tracking heart sound components are disclosed. An exemplary medical-device system includes a data receiver to receive heart sound information, and a heart sound recognition circuit to generate a representative heart sound segment such as an ensemble average of a selected portion of heart sound segments. The heart sound recognition circuit recognizes a heart sound component from the representative heart sound segment using a time-based tracking algorithm, and evaluates a performance index of the time-based heart sound tracking algorithm. Based on the performance index, a decision can be made whether or not to switch to a spectral-based tracking algorithm to recognize the heart sound component from the representative heart sound segment. A physiologic event detector can detect a cardiac event using the recognized heart sound component.

### CLAIM OF PRIORITY

This application claims the benefit of U.S. Provisional Application No. 63/599,867, filed on Nov. 16, 2023, which is hereby incorporated by reference in its entirety.

### TECHNICAL FIELD

This document relates generally to medical systems, and more particularly, to systems, devices and methods for recognizing and tracking heart sounds in a subject.

### BACKGROUND

Heart sounds are generally associated with mechanical vibration of a heart and blood flow through the heart. Heart sounds recur with each cardiac cycle and are separated and classified according to the activity associated with the vibration. Historically, heart sounds were assessed by humans and thus only audible portion of the vibration was used. Devices can now assess full spectrum of cardiac vibrations which include both audible and subaudible components, thus the term “sound” in this document refers to the full spectrum of vibrations. Typically, heart sounds sensed from a subject may include several components within a cardiac cycle, including a first (S1), a second (S2), a third (S3), or a fourth (S4) heart sound. S1 is associated with the vibrations produced by the heart during tensing of the mitral valve. S2 is produced by closure of the aortic and pulmonary valves, and marks the beginning of diastole. S3 is produced by early diastolic vibrations corresponding to passive ventricular filling during diastole, when the blood rushes into the ventricles. S4 is produced by late diastolic vibrations corresponding to active ventricular filling when the atria contract and push the blood into the ventricles. In a healthy subject, S3 is usually faint and S4 is rarely audible. However, a pathologic S3 or S4 may be higher pitched and louder.

Heart sounds have been used to assess cardiac systolic and diastolic functions. Systole is the contraction or a period of contraction of the heart that causes blood to be forced out of the heart such as the ventricles and into the aorta and pulmonary artery. Diastole is the relaxation or a period of relaxation of the heart during which the blood flows back into the heart such as the ventricles. Patients with cardiac diseases may have deteriorated systolic or diastolic functions. For example, congestive heart failure (CHF) occurs when the heart is unable to supply enough blood to maintain a healthy physiologic state.

Implantable medical devices (IMDs) have been used to monitor patients with cardiac disease, such as to detect cardiac events leading to worsening heart failure (WHF). An IMD may sense physiologic signals from a patient, and deliver electrostimulation therapy to improve cardiac performance in CHF patients. Frequent patient monitoring via an IMD may help identify patients having an elevated risk of developing future heart failure events, ensure timely treatment, reduce heart failure hospitalization, improve patient outcome, and reduce healthcare cost.

### OVERVIEW

An ambulatory medical device (AMD), such as an implantable medical device (IMD), a subcutaneous medical device, a wearable medical device, or other external medical device, may be used to monitor cardiac patient. An AMD may sense electrical or mechanical activities of the heart via sensing electrodes and/or physiologic sensors, and detect cardiac events such as cardiac arrhythmias or WHF. An IMD may include a pulse generator capable of generating and delivering electrostimulation therapy to the heart or other excitable tissue (e.g., neural targets) to restore or improve cardiac performance in a CHF patient, or to correct cardiac arrhythmia. For example, a detection of cardiac arrhythmia may trigger cardiac pacing or electric shock, or a detection of a WHF event may trigger electrostimulation therapy, such as a resynchronization therapy (CRT) to correct cardiac dyssynchrony in patients with heart failure.

An AMD can detect a cardiac event using heart sounds detected from a patient. For example, S1 and/or S2 may be used to detect cardiac arrhythmia such as supraventricular tachycardia or ventricular tachycardia. Pulmonary fluid accumulation in CHF patients may cause elevated ventricular filling pressure and diastolic dysfunction, resulting in pathologically louder S3. Forceful atrial contraction to overcome an abnormally stiff ventricle in a CHF patient may produce profound S4. Therefore, monitoring S3 or S4 may help determining patient diastolic dysfunction, detecting a WHF event, or assessing patient risk of developing future WHF.

Ambulatory heart sounds detection involves placing a heart sound sensor at an epicutaneous, subcutaneous, submuscular, intramuscular, or substernal location at or near the heart. The heart sound sensor, such as an accelerometer, may be included within an IMD for implantation, or associated with an implantable lead for epicardial or endocardial placement. S1 and S2 heart sounds generally have a frequency within a range of approximately 10-250 Hz. With a wide interpersonal variability and shifts secondary to technical equipment, S2 generally has a higher frequency than S1. For example, most of S1 power fall within approximately 10-50 Hz and most of S2 fall within approximately 20-70 Hz, Early diastole sound S3 produced by rapid filling of dilated ventricles, and late diastole sound S4 produced by contraction of the left atrium against a non-compliant left ventricle, when present, generally have lower intensity and lower frequencies.

A heart sound component (e.g., S1, S2, S3, or S4) may be recognized using a time-domain, amplitude-based approach, which involves detecting peak amplitude or a variation thereof, such as peak signal power or root-mean-square (RMS) value, of a heart sound signal within a heart sound detection window that defines a time interval for detecting a heart sound component of interest. The timing of the maximum amplitude (or the signal power) is then identified as the timing location of the heart sound component of interest. However, often times multiple peaks can be present within the heart sound signal of an individual heart sound window, and the major peak (the one with the largest amplitude) may not always represent an actual heart sound component. As such, the time-domain, amplitude-based approach may sometimes mistakenly recognize a spurious peak as a target heart sound component. The time-domain, amplitude-based approach may also be sensitive to electromagnetic or physiological noise or interferences. At a low signal-to-noise ratio (SNR), timing information of a heart sound component such as S1 or S2 may not be accurately determined. This may cause further errors in the detections of other heart sound components (e.g., S3 or S4) or heart sound-based cardiac time intervals that are measured in reference to the timings of S1 or S2, such as pre-ejection period (PEP, an interval between QRS onset and S1), a systolic timing interval (STI, an interval between QRS onset and S2), a left-ventricular ejection time (LVET, an interval between S1 and S2), or a diastolic timing interval (DTI, an interval between S2 and QRS onset of the next cardiac cycle). When heart sound timings and/or cardiac time intervals are used to detect a cardiac event detections (e.g., WHF events), the detection performance may be reduced. Other factors, such as inaccurate estimate of heart rates (such as during atrial arrhythmia such as atrial fibrillation), flipping of the implantable medical device, or malposition or twisting of the sensing lead system, may also lead to errors in time-domain, amplitude-based heart sound recognition.

An alternative approach for detecting and tracking a heart sound component is based on frequency or spectral features of a heart sound signal. Complexity measures such as Shannon's entropy or other entropy measures may be obtained from a power spectrum of a heart sound signal (or a portion thereof), and used for recognizing a heart sound component of interest, as described in Goftari et al. U.S. Patent Application No. 63/432,649 entitled “ENTROPY BASED HEART SOUND TRACKING.” Such spectral entropy-based approach quantifies the complexities (or non-uniformities) of power spectral magnitudes of a heart sound signal or a portion thereof such as within a cardiac cycle. The spectral entropy-based heart sound recognition combines the advantages of frequency-domain spectral analysis and entropy measures, both of which can be less susceptible to noise and interferences from various sources than the time-domain, amplitude-based heart sound detection method. In particular, for example, the spectral entropy-based approach can be more immune to sporadic time-domain high-amplitude noise which is a leading cause to spurious detections. However, compared to time-domain, amplitude-based recognition method, the spectral entropy-based method is generally more computationally intensive, and may require more computing resources and consume more power of the implantable device.

The present inventors have recognized an unmet need for devices and methods of more robust heart sound recognition and tracking, while keeping the power consumption and computation complexity under control. A heart sound recognition technique as such may improve accuracy and efficiency of detecting various cardiac events (e.g., a variety of cardiac arrhythmia, or worsening heart failure). The present document discusses systems, devices, and methods for assessing qualities of various heart sound recognition algorithms, identifying therefrom an appropriate algorithm for use in detecting a cardiac event. An exemplary medical-device system comprises a data receiver circuit to receive heart sound information, and a heart sound recognition circuit to generate a representative heart sound segment within a cardiac cycle, such as an ensemble average of a selected portion of heart sound segments from multiple cardiac cycles. The heart sound recognition circuit can use a time-based tracking algorithm to recognize a heart sound component interest from the representative heart sound segment, and evaluate a performance index of the time-based heart sound tracking algorithm based on morphological or timing information of the recognized heart sound component. Based on the performance index, the heart sound recognition circuit can determine whether or not to switch to a spectral-based tracking algorithm to further recognize the heart sound component. The heart sound component thus recognized can be used to detect a cardiac event such as a worsening heart failure (WHF) event.

Example 1 is a medical-device system that includes: a data receiver circuit configured to receive heart sound information over multiple cardiac cycles; and a heart sound recognition circuit configured to: generate a representative heart sound segment within a cardiac cycle using at least a portion of the received heart sound information; recognize a heart sound component from the representative heart sound segment using a time-based heart sound tracking algorithm; evaluate a performance index of the time-based heart sound tracking algorithm using morphological or timing information of the recognized heart sound component; and based on the evaluated performance index, determine whether or not to switch to a spectral-based heart sound tracking algorithm to recognize the heart sound component from the representative heart sound segment.

In Example 2, the subject matter of Example 1 optionally includes the time-based heart sound tracking algorithm that can recognize the heart sound component using amplitude and timing information of the representative heart sound segment.

In Example 3, the subject matter of any one or more of Examples 1-2 optionally includes the spectral-based heart sound tracking algorithm that can recognize the heart sound component using one or more spectral entropy values calculated from the representative heart sound segment.

In Example 4, the subject matter of any one or more of Examples 1-3 optionally includes the portion of the received heart sound information that can include a plurality of heart sound segments corresponding to respective heart beats, wherein the heart sound recognition circuit is configured to generate the representative heart sound segment using an ensemble average of at least a subset of the plurality of heart sound segments.

In Example 5, the subject matter of Example 4 optionally includes the data receiver circuit that can be configured to receive information about instantaneous heart rates of the heart beats that correspond to the plurality of heart sound segments, wherein the at least the subset of the plurality of heart sound segments being used for generating the representative heart sound segment corresponds to heart beats falling within a pre-determined heart rate range.

In Example 6, the subject matter of Example 5 optionally includes, wherein to generate the representative heart sound segment, the heart sound recognition circuit is configured to: sort the plurality of heart sound segments according to a specific order of the instantaneous heart rates of the heart beats corresponding to the plurality of heart sound segments; identify, from the sorted plurality of heart sound segments, at least a first group of heart sound segments corresponding to first heart beats, and a second group of heart sound segments corresponding to second heart beats having different heart rates than the first heart beats; determine a first morphology similarity metric of the first group of heart sound segments, and a second morphology similarity metric of the second group of heart sound segments; select a group between the first group and the second group based on one or more of the first morphology similarity metric or the second morphology similarity metric; and generate the representative heart sound segment using an ensemble average of the selected group of heart sound segments.

In Example 7, the subject matter of Example 6 optionally includes the first group and the second group each having at least a specific minimum number of heart sound segments.

In Example 8, the subject matter of any one or more of Examples 6-7 optionally includes the first heart beats corresponding to the first group of heart sound segments and the second heart beats corresponding to the second group of heart sound segments each having respective instantaneous heart rates falling below a heart rate threshold.

In Example 9, the subject matter of any one or more of Examples 6-8 optionally includes the first heart beats corresponding to the first group of heart sound segments and the second heart beats corresponding to the second group of heart sound segments each having respective instantaneous heart rates satisfying a heart rate variability or range requirement.

In Example 10, the subject matter of any one or more of Examples 6-9 optionally includes the plurality of heart sound segments that can be sorted according to an ascending order of the instantaneous heart rates of the heart beats, wherein the second heart beats corresponding to the second group of heart sound segments have higher heart rates than the first heart beats corresponding to the first group of heart sound segments, wherein to select between the first group and the second group includes: to select the first group if (i) the first morphology similarity metric is above a first threshold, or (ii) both the first and the second morphology similarity metrics are below respective thresholds and the first morphology similarity metric is greater than the second morphology similarity metric; and to select the second group if (i) the first morphology similarity metric is below the first threshold and the second morphology similarity metric is above a second threshold, or (ii) both the first and the second morphology similarity metrics are below the respective thresholds and the second morphology similarity metric is greater than the first morphology similarity metric.

In Example 11, the subject matter of any one or more of Examples 4-10 optionally includes the heart sound recognition circuit that can be configured to evaluate the performance index of the time-based heart sound tracking algorithm using a morphology similarity metric among the at least the subset of the plurality of heart sound segments being used for generating the representative heart sound segment.

In Example 12, the subject matter of Example 11 optionally includes, wherein to evaluate the performance index of the time-based heart sound tracking algorithm includes further using a heart sound tracking stability indicative of variability of timings of a heart sound component respectively determined from the at least the subset of the plurality of heart sound segments being used for generating the representative heart sound segment, wherein to determine whether or not to switch to the spectral-based heart sound tracking algorithm, the heart sound recognition circuit is configured to: determine not to switch to the spectral-based heart sound tracking algorithm to recognize the heart sound component from the representative heart sound segment if (i) the morphology similarity metric exceeds a similarity threshold, and (ii) the heart sound tracking stability is below a variability threshold; and determine to switch to the spectral-based heart sound tracking algorithm to recognize the heart sound component from the representative heart sound segment if (i) the morphology similarity metric is no greater than the similarity threshold, or (ii) the heart sound tracking stability exceeds the variability threshold.

In Example 13, the subject matter of Example 12 optionally includes the heart sound tracking stability that can be indicative of variability of timings of S2 component, wherein the heart sound recognition circuit is configured to determine aortic stenosis (AS) indices indicating a presence or severity of AS for each of the at least the subset of the plurality of heart sound segments being used for generating the representative heart sound segment, and to determine the heart sound tracking stability using a variability of the determined AS indices.

In Example 14, the subject matter of any one or more of Examples 4-13 optionally includes, wherein to evaluate the performance index of the time-based heart sound tracking algorithm, the heart sound recognition circuit is configured to: generate a heart sound timing trend comprising a time series of timings of the heart sound component identified from the at least the subset of the plurality of heart sound segments using the time-based heart sound tracking algorithm; generate a heart rate trend comprising a time series of instantaneous heart rates of the heart beats corresponding to the at least the subset of the plurality of heart sound segments; and evaluate the performance index of the time-based heart sound tracking algorithm using a concordance metric between the heart sound timing trend and the heart rate trend, wherein to determine whether or not to switch to the spectral-based heart sound tracking algorithm, the heart sound recognition circuit is configured to: determine not to switch to the spectral-based heart sound tracking algorithm to recognize the heart sound component from the representative heart sound segment if the concordance metric falls below a threshold; and determine to switch to the spectral-based heart sound tracking algorithm to recognize the heart sound component from the representative heart sound segment if the concordance metric exceeds the threshold.

In Example 15, the subject matter of any one or more of Examples 1-14 optionally includes a physiological event detector that can be configured to a detect a cardiac event using the recognized heart sound component.

Example 16 is a method of detecting a cardiac event, comprising steps of: receiving heart sound information over multiple cardiac cycles; generating a representative heart sound segment within a cardiac cycle using at least a portion of the received heart sound information; recognizing a heart sound component from the representative heart sound segment using a time-based heart sound tracking algorithm; evaluating a performance index of the time-based heart sound tracking algorithm using morphological or timing information of the recognized heart sound component; based on the evaluated performance index, determining whether or not to switch to a spectral-based heart sound tracking algorithm to recognize the heart sound component from the representative heart sound segment; and detecting the cardiac event using the recognized heart sound component.

In Example 17, the subject matter of Example 16 optionally includes the time-based heart sound tracking algorithm that can recognize the heart sound component using amplitude and timing information of the representative heart sound segment, wherein the spectral-based heart sound tracking algorithm recognizes the heart sound component using one or more spectral entropy values calculated from the representative heart sound segment.

In Example 18, the subject matter of any one or more of Examples 13-17 optionally includes the portion of the received heart sound information that can include a plurality of heart sound segments corresponding to respective heart beats, wherein generating the representative heart sound segment includes: sorting the plurality of heart sound segments according to a specific order of instantaneous heart rates of the heart beats corresponding to a plurality of heart sound segments; identifying, from the sorted plurality of heart sound segments, at least a first group of heart sound segments corresponding to first heart beats, and a second group of heart sound segments corresponding to second heart beats having different heart rates than the first heart beats; determining a first morphology similarity metric of the first group of heart sound segments, and a second morphology similarity metric of the second group of heart sound segments; selecting a group between the first group and the second group based on one or more of the first morphology similarity metric or the second morphology similarity metric; and generating the representative heart sound segment using an ensemble average of the selected group of heart sound segments.

In Example 19, the subject matter of Example 18 optionally includes the first group and the second group each having at least a specific minimum number of heart sound segments, wherein the first heart beats corresponding to the first group of heart sound segments and the second heart beats corresponding to the second group of heart sound segments each have respective instantaneous heart rates falling below a heart rate threshold and satisfying a heart rate variability or range requirement.

In Example 20, the subject matter of any one or more of Examples 18-19 optionally includes the plurality of heart sound segments that can be sorted according to an ascending order of the instantaneous heart rates of the heart beats, wherein the second heart beats corresponding to the second group of heart sound segments have higher heart rates than the first heart beats corresponding to the first group of heart sound segments, wherein selecting between the first group and the second group includes: selecting the first group if (i) the first morphology similarity metric is above a first threshold, or (ii) both the first and the second morphology similarity metrics are below respective thresholds and the first morphology similarity metric is greater than the second morphology similarity metric; and selecting the second group if (i) the first morphology similarity metric is below the first threshold and the second morphology similarity metric is above a second threshold, or (ii) both the first and the second morphology similarity metrics are below the respective thresholds and the second morphology similarity metric is greater than the first morphology similarity metric.

In Example 21, the subject matter of any one or more of Examples 16-20 optionally includes the portion of the received heart sound information that can include a plurality of heart sound segments corresponding to respective heart beats, wherein evaluating the performance index of the time-based heart sound tracking algorithm includes using (i) a morphology similarity metric among at least a subset of the plurality of heart sound segments being used for generating the representative heart sound segment and (ii) a heart sound tracking stability indicative of variability of timings of a heart sound component respectively determined from the at least the subset of the plurality of heart sound segments being used for generating the representative heart sound segment, wherein determining whether or not to switch to the spectral-based heart sound tracking algorithm includes: determining not to switch to the spectral-based heart sound tracking algorithm to recognize the heart sound component from the representative heart sound segment if (i) the morphology similarity metric exceeds a similarity threshold, and (ii) the heart sound tracking stability is below a variability threshold; and determining to switch to the spectral-based heart sound tracking algorithm to recognize the heart sound component from the representative heart sound segment if (i) the morphology similarity metric is no greater than the similarity threshold, or (ii) the heart sound tracking stability exceeds the variability threshold.

In Example 22, the subject matter of any one or more of Examples 16-21 optionally includes the portion of the received heart sound information that can include a plurality of heart sound segments corresponding to respective heart beats, wherein evaluating the performance index of the time-based heart sound tracking algorithm includes: generating a heart sound timing trend comprising a time series of timings of the heart sound component identified from at least a subset of the plurality of heart sound segments using the time-based heart sound tracking algorithm; generating a heart rate trend comprising a time series of instantaneous heart rates of the heart beats corresponding to the at least the subset of the plurality of heart sound segments; and evaluating the performance index of the time-based heart sound tracking algorithm using a concordance metric between the heart sound timing trend and the heart rate trend, wherein determining whether or not to switch to the spectral-based heart sound tracking algorithm includes: determining not to switch to the spectral-based heart sound tracking algorithm to recognize the heart sound component from the representative heart sound segment if the concordance metric falls below a threshold; and determining to switch to the spectral-based heart sound tracking algorithm to recognize the heart sound component from the representative heart sound segment if the concordance metric exceeds the threshold.

This Overview provides some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally an example of a heart sound-based physiologic event detection system that recognizes a heart sound component, and uses the recognized heart sound component to detect a target physiologic event.

FIG. 3 illustrates generally an example of generating a heart sound ensemble using a selected subset of pre-screened, sorted heart sound segments.

FIG. 4 is a flowchart illustrating an example method of selecting from candidate groups a group of pre-screened, sorted heart sound segments based on morphology similarity among the heart sound segments.

FIG. 5A illustrates a heart sound tracking assessment circuit that generates a heart sound recognition performance index using a heart sound morphology similarity metric and a heart sound tracking stability.

FIG. 5B is a flowchart illustrating an example method of assessing the performance of the time-based tracker using the heart sound tracking assessment circuit illustrated in FIG. 5A, and using the assessed performance to determine whether or not to switch to the spectral-based tracker to recognize a heart sound component of interest.

FIG. 6A illustrates a heart sound tracking assessment circuit that generates a heart sound recognition performance index using a concordance metric between a heart rate trend and a heart sound timing trend.

FIG. 6B is a flowchart illustrating an example method for assessing the performance of the time-based tracker using the heart sound tracking assessment circuit illustrated in FIG. 6A, and using the assessed performance to determine whether or not to switch to the spectral-based tracker to recognize a heart sound component of interest.

FIGS. 7A-7B illustrates an example of quantifying heart sound tracking stability based on an aortic stenosis indication, and using the same to determine whether or not to switch to a spectral-based tracker to recognize a heart sound component of interest.

FIGS. 8A-8B illustrates an example of determining a concordance between heart rates and S2 timings, and using the same to determine whether or not to switch to a spectral-based tracker to recognize a heart sound component of interest.

FIG. 9 illustrates generally an example of detecting worsening heart failure (WHF) based at least in part on S3 heart sounds recognized and tracked in accordance with various examples described in this document.

FIG. 10 illustrates generally an example of a method for recognizing and tracking a heart sound component and using the same to detect a physiologic event.

FIG. 11 illustrates generally a block diagram of an example machine upon which any one or more of the techniques (e.g., methodologies) discussed herein may perform.

### DETAILED DESCRIPTION

Disclosed herein are systems, devices, and methods for recognizing and tracking a heart sound component using one or more distinct recognition and tracking algorithms. An exemplary medical-device system includes a data receiver to receive heart sound information, and a heart sound recognition circuit to generate a representative heart sound segment, such as an ensemble average of a selected portion of heart sound segments from multiple cardiac cycles. The heart sound recognition circuit recognizes a heart sound component from the representative heart sound segment using a time-based tracking algorithm, and evaluates a performance index of the time-based heart sound tracking algorithm. Based on the performance index, a decision can be made whether or not to switch to a spectral-based tracking algorithm to further recognize the heart sound component from the representative heart sound segment. A physiologic event detector can detect a cardiac event using the recognized heart sound component.

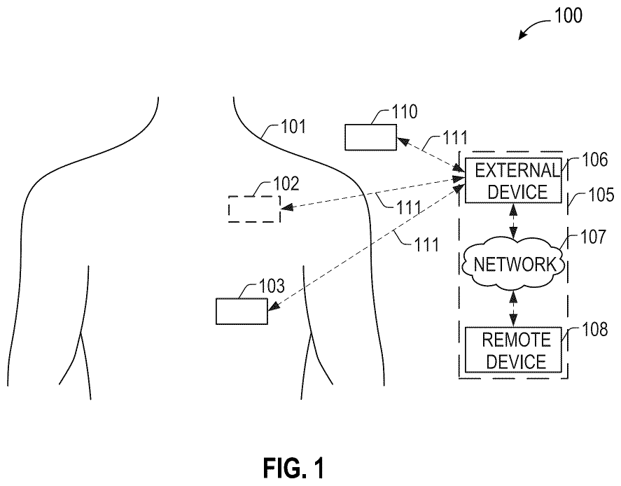

FIG. 1 illustrates generally an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 may include one or more ambulatory medical devices, an external system 105, and a communication link 111 providing for communication between the one or more ambulatory medical devices and the external system 105. The one or more ambulatory medical devices may include an implantable medical device (IMD) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 may include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 may include a monitor implanted, for example, subcutaneously in the chest of patient 101, the IMD 102 including a housing containing circuitry and, in certain examples, one or more sensors, such as a temperature sensor, etc.

The IMD 102 may include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy may include delivery of one or more drugs to the patient 101, such as using the IMD 102 or one or more of the other ambulatory medical devices, etc. In some examples, therapy may include cardiac resynchronization therapy for rectifying dyssynchrony and improving cardiac function in heart failure patients. In other examples, the IMD 102 may include a drug delivery system, such as a drug infusion pump to deliver drugs to the patient for managing arrhythmias or complications from arrhythmias, hypertension, or one or more other physiologic conditions. In other examples, the IMD 102 may include one or more electrodes configured to stimulate the nervous system of the patient or to provide stimulation to the muscles of the patient airway, etc.

The WMD 103 may include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

In an example, the IMD 102 or the WMD 103 may include or be coupled to an implantable or wearable sensor to sense a heart sound signal, and include a heart sound recognition circuit to recognize one or more heart sound components such as S1, S2, S3, or S4 based on a spectral entropy time series derived from the sensed heart sound signal. Also included in the IMD 102 or the WMD 103 is a heart sound-based event detector circuit that can detect a physiologic event (e.g., a cardiac arrhythmia episode, or a worsening heart failure (WHF) event) based at least on a heart sound metric of the detected one or more heart sound component. Examples of such heart sound metric may include an amplitude, or timing of the heart sound component within a cardiac cycle relative to a fiducial point. In some examples, at least a portion of the heart sound recognition circuit and/or the heart sound-based event detector circuit may be implemented in and executed by the external system 105.

The external system 105 may include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This may include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information may include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radiofrequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 may include an external device 106 in proximity of the one or more ambulatory medical devices, and a remote device 108 in a location relatively distant from the one or more ambulatory medical devices, in communication with the external device 106 via a communication network 107. Examples of the external device 106 may include a medical device programmer. The remote device 108 can be configured to evaluate collected patient or patient information and provide alert notifications, among other possible functions. In an example, the remote device 108 may include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server may include a memory device to store the data in a patient database. The server may include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications may include a Web page update, phone or pager call, E-mail, SMS, text or “Instant” message, as well as a message to the patient and a simultaneous direct notification to emergency services and to the clinician. Other alert notifications are possible. The server may include an alert prioritizer circuit configured to prioritize the alert notifications. For example, an alert of a detected physiologic event can be prioritized using a similarity metric between the physiological data associated with the detected physiologic event to physiological data associated with the historical alerts.

The remote device 108 may additionally include one or more locally configured clients or remote clients securely connected over the communication network 107 to the server. Examples of the clients may include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiologic events to a system user, such as the patient or a clinician, or to a process including, for example, an instance of a computer program executable in a microprocessor. In an example, the process may include an automated generation of recommendations for anti-arrhythmic therapy, or a recommendation for further diagnostic test or treatment. In an example, the external device 106 or the remote device 108 may include a respective display unit for displaying the physiologic or functional signals, or alerts, alarms, emergency calls, or other forms of warnings to signal the detection of arrhythmias. In some examples, the external system 105 may include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” may include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 may include a local external implantable medical device programmer. The external system 105 may include a remote patient management system that can monitor patient status or adjust one or more therapies such as from a remote location.

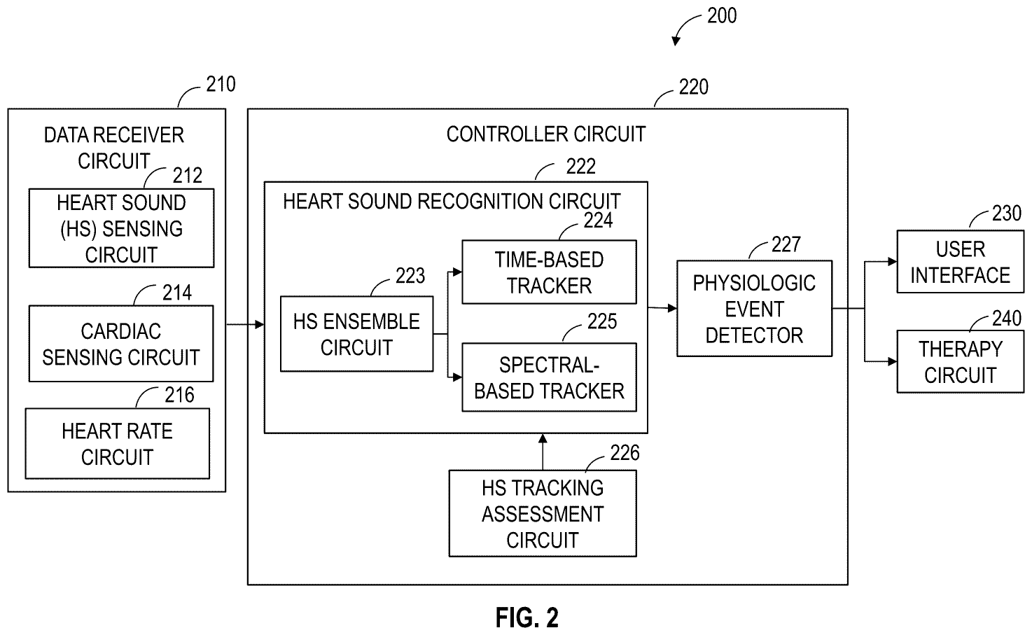

FIG. 2 illustrates generally an example of a heart sound-based physiologic event detection system 200 that can recognize a heart sound component of interest, to detect a physiologic event, such as cardiac arrhythmia or a worsening heart failure (WHF) event, using information about the recognized heart sound component. The heart sound-based physiologic event detection system 200 may include one or more of a data receiver circuit 210, a controller circuit 220, a user interface 230, and a therapy circuit 240. At least a portion of the heart sound-based physiologic event detection system 200 may be implemented in the IMD 102, the WMD 103, or the external system 105 such as one or more of the external device 106 or the remote device 108.

The data receiver circuit 210 may receive physiologic information from a patient. In an example, the data receiver circuit 210 may include a sense amplifier circuit configured to sense a physiologic signal from a patient via a physiologic sensor, such as an implantable, wearable, or otherwise ambulatory sensor or electrodes associated with the patient. The sensor may be incorporated into, or otherwise associated with an ambulatory device such as the IMD 102 or the WMD 103. In some examples, the physiologic signals sensed from a patient may be stored in a storage device, such as an electronic medical record (EMR) system. The data receiver circuit 210 may receive the physiologic signal from the storage device, such as in response to a user command or a triggering event. By way of example and not limitation and as illustrated in FIG. 2, the data receiver circuit 210 may include one or more of a heart sound sensing circuit 212, a cardiac sensing circuit 214, or a heart rate circuit 216. The heart sound sensing circuit 212 can receive heart sound information, such as a heart sound signal sensed from the patient. In an example, the heart sound sensing circuit 212 may be coupled to a heart sound sensor to sense a body motion/vibration signal indicative of cardiac vibration, which is correlated to or indicative of heart sounds. The heart sound sensor may take the form of an accelerometer, an acoustic sensor, a microphone, a piezo-based sensor, or other vibrational or acoustic sensors. The accelerometer can be a one-axis, a two-axis, or a three-axis accelerometer. Examples of the accelerometer may include flexible piezoelectric crystal (e.g., quartz) accelerometer or capacitive accelerometer, fabricated using micro electro-mechanical systems (MEMS) technology. The heart sound sensor may be included in the IMD 102 or the WMD 103, or disposed on a lead such as a part of the lead system associated with the IMD 102 or the WMD 103. In an example, an accelerometer (or other sensor types) may sense an epicardial or endocardial acceleration (EA) signal from a portion of a heart, such as on an endocardial or epicardial surface of one of a left ventricle, a right ventricle, a left atrium, or a right atrium. The EA signal may contain components corresponding to various heart sound components such as one or more of S1, S2, S3, or S4.

The cardiac sensing circuit 214 can sense a cardiac electrical signal. Examples of the cardiac electrical signal may include surface electrocardiography (ECG) sensed from electrodes placed on the body surface, subcutaneous ECG sensed from electrodes placed under the skin, intracardiac electrogram (EGM) sensed from the one or more implantable electrodes. The sensing electrodes can be included in or communicatively coupled to the IMD 102 or the WMD 103. The heart rate circuit 216 can detect heart rate of the patient, such as from the signal sensed by the cardiac sensing circuit 214. Alternatively, the heart rate (or pulse rate) can be detected from a cardiac mechanical signal. As to be discussed further below, one or more of the cardiac electrical signal or the heart rate information may be used by the controller circuit to construct a representative heart sound segment from which a spectral entropy time series can be derived.

In some examples, the data receiver circuit 210 may receive other physiological or functional signals including, for example, physical activity signal, posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide (BNP), blood panel, sodium and potassium levels, glucose level and other biomarkers and bio-chemical markers, among others.

The controller circuit 220 can recognize and track a heart sound component, and detect a physiologic event based at least one the heart sound component. The controller circuit 220 be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The controller circuit 220 may include circuit sets comprising one or more other circuits or sub-circuits, such as a heart sound recognition circuit 222, a heart sound tracking assessment circuit 226, and a physiologic event detector 227. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The heart sound recognition circuit 222 can preprocess the heart sound signal received from the heart sound sensing circuit 212. In an example, the heart sound recognition circuit 222 may include a filter or a filter bank to remove or attenuate one or more of low-frequency signal baseline drift, high frequency noise, or other unwanted frequency contents. In an example, the heart sound segment may be band-pass filtered to a frequency range of approximately 5-90 Hz, or approximately 9-90 Hz. In an example, the filter may include a double or higher-order differentiator configured to calculate a double or higher-order differentiation of the heart sound signal.

The heart sound recognition circuit 222 may recognize a heart sound component of interest (e.g., one or more of S1, S2, S3, or S4) from the preprocessed heart sound signal, Cardiac electrical signal sensed by the cardiac sensing circuit 214 and/or heart rate information sensed by the heart rate circuit 216 may be used to assist in recognizing the heart sound component. To improve the heart sound signal quality and the heart sound recognition accuracy, the heart sound recognition circuit 222 may generate a representative heart sound segment within a cardiac cycle using at least a portion of the received heart sound signal. In an example, the representative heart sound segment may be generated using an ensemble average of a selected group of heart sound segments. A heart sound ensemble comprises heart sound segments in respective cardiac cycles. In an example, the heart sound ensemble circuit 323 may use the cardiac electrical signal (from the cardiac sensing circuit 214) concurrently sensed with the heart sound signal. The heart sound ensemble circuit 323 may detect cardiac cycles as time intervals between two consecutive ventricular activations such as R waves or QRS complexes on the ECG or intracardiac EGM signal. The heart sound ensemble circuit 323 may align the heart sound segments with respect to respective fiducial points over multiple cardiac cycles. The fiducial points may include, for example, ventricular activation timings on a ECG or EGM signal over multiple cardiac cycles.

The heart sound ensemble circuit 323 may screen the heart sound segments over multiple cardiac cycles, and select a subset of the heart sound segments that meet specific criteria to form a heart sound ensemble. The selection criteria may include one or more of a heart rate, a heart rate variability or range, an activity level, a respiration rate, or time of a day, among others. In one example, the subset of the heart sound segments may be selected to correspond to heart rates falling below a predetermined or user-set heart rate threshold (e.g., 100-120 bpm). In another example, the subset of the heart sound segments may be selected to correspond to heart rates or cycle lengths falling within a specified range, such as substantially the same cycle length within a margin of +/−100 milliseconds, or substantially the same heart rate within a margin (heart rate variability or range) of +/−5 beats per minute (bpm) in one example, or +/−10 bpm in another example.

In addition or alternative to the heart rate and heart rate variability or range criteria, other requirements may be imposed on the selected subset of the heart sound segments. For example, the subset of the heart sound segments may be selected to correspond to a physical activity level of the subject falling within a specified range, such as substantially the same physical activity level within a specified margin. Additionally or alternatively, the subset of the heart sound segments may be selected to correspond to a respiratory rate of the subject falling within a specified range, such as substantially the same respiratory rate within a specified margin. In yet another example, the heart sound signal may be sensed during a specified time period of a day, and the heart sound ensemble circuit 323 may select the subset of the heart sound segments that are sensed during substantially the same time period of a day within a specified margin.

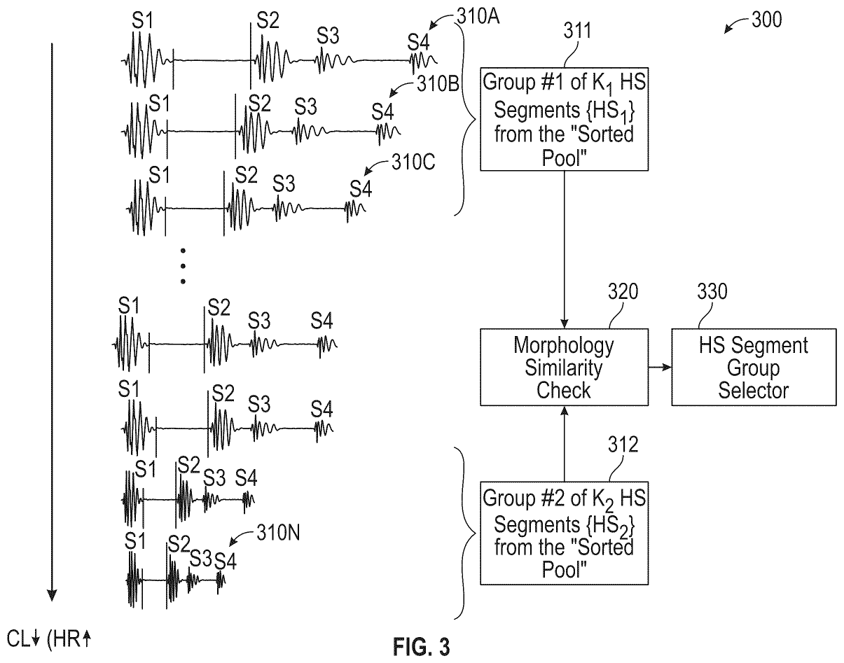

In some examples, the heart sound ensemble circuit 323 may sort the heart sound segments over multiple cardiac cycles according to a specific order of heart rates of the heart beats corresponding to the heart sound segments. From the “sorted pool” of heart sound segments, at least a first group of heart sound segments corresponding to first heart beats, denoted by {HS₁}, and a second group of heart sound segments corresponding to second heart beats, denoted by {HS₂}, may be identified. The second heart beats have different heart rates than the first heart beats. Morphology similarities may be evaluated among the heart sound segments in each of the groups, resulting in a first morphology similarity metric MS₁ among the first group of heart sound segments {HS₁}, and a second morphology similarity metric MS₂ among the second group of heart sound segments {HS₂}. Referring now to FIG. 3, diagram 300 illustrates an example of creating a heart sound ensemble using a selected subset of pre-screened, “sorted pool” of heart sound segments {310A, 310B, 310C, . . . 310N}. In this non-limiting example, the heart sound segments are sorted according to an ascending order of instantaneous heart rates of the heart beats corresponding to the heart sound segments {310A, 310B, 310C, . . . 310N}. The heart sound segments in the sorted pool are pre-screened such that they have met the screening criteria based on one or more of a heart rate, a heart rate variability or range, a respiration rate, or an activity level, among others as described above. Different groups of heart sound segments corresponding to heart beats with different heart rates may be assembled using respective different portions of the sorted pool of heart sound segments. By way of example and not limitation, FIG. 3 shows a first group 311 {HS₁} including first K₁ heart sound segments in the sorted pool, and a second group 312 {HS₂} including last K₂ heart sound segments in the sorted pool. The first group {HS₁} correspond to first K₁ heart beats, and the second group of heart sound segments {HS_{HR2}} correspond to last K₂ heart beats having higher heart rates than the first K₁ heart beats. The beat numbers, K₁ and K₂, can be a user-specified or programmable number within a range, such as between 8 and 16 beats in a non-limiting example. In an example, at least eight qualified heart beats that satisfy the heart rate and/or heart rate variability requirement are required for each of the first and second groups. The first K₁ heart beats and the last K₂ heart beats each satisfy the heart rate and heart rate variability requirements, such as the instantaneous heart rates in each group falling below a heart rate threshold (e.g., 120 bpm in an example), and the heart rate variability in each group falling below a variability threshold. In an example, the heart rate variability may be computed as a difference ΔHR between the maximum instantaneous heart rate and the minimum instantaneous heart rate among the heart beats of a group. The heart rate difference of the first K₁ heart beats, ΔHR₁, and the heart rate difference of the last K₂ heart beats, ΔHR₁, each is required to be less than a threshold ΔHR_{TH} (e.g., 10 bpm in an example).

The heart sound ensemble circuit 223 may include a heart sound segment group selector 330 to select a group between the first group {HS₁} and second group {HS₂}, and generate the representative heart sound segment using an ensemble average of the heart sound segments of the selected group. The selection may be based on a morphology similarity check 320, where one or more of a first morphology similarity metric (MS₁) among the heart sound segments in the first group {HS₁}, or a second morphology similarity metric (MS₂) among the heart sound segments in the second group {HS₂}, may be determined. The morphology similarity metric represents a similarity of waveforms of heart sound portions or heart sound components respectively extracted from heart sound segments, such as those from the first group {HS₁} or the second group {HS₂}. In an example, the morphology similarity metric may be computed using heart sound waveform portions within a specified window, such as a S2 detection window. The S2 detection window may be defined with respect to a fiducial point such as a ventricular activation timing (e.g., R wave on an ECG waveform). In an example, the morphology similarity metric may be computed as accumulated deviations or root-mean-squared (RMS) values of heart sound waveforms within the same group. In another example, the morphology similarity metric may be computed as a correlations between morphological features of the heart sound waveforms within the same group.

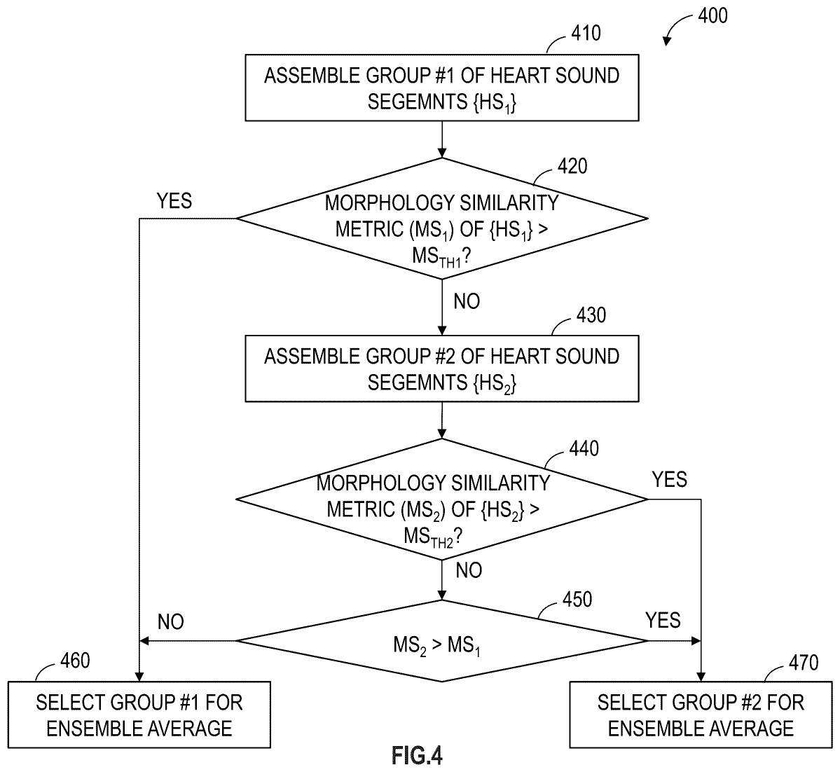

Referring now to FIG. 4, the flowchart represents an example method 400 of selecting a group of pre-screened, sorted heart sound segments based on morphology similarity among the heart sound segments. The method 400 may be used by the heart sound ensemble circuit 323 to select between the first group {HS₁} and second group {HS₂} based on one or more of the first morphology similarity metric MS₁ or the second morphology similarity metric MS₂. At step 410, the first group {HS₁} may be assembled from the sorted pool of the heart sound segments, as illustrated in FIG. 3. At step 420, a morphology similarity metric MS₁ of {HS₁} is computed, and compared to a threshold value MS_{TH1}. If MS₁ is greater than MS_{TH1}, then at step 460 the first group {HS₁} is deemed satisfactory, and can be selected as the heart sound ensemble for computing the ensemble average. If MS₁ is no greater than MS_{TH1}, then at step 430 the second group {HS₂} may be assembled from the sorted pool of the heart sound segments, as illustrated in FIG. 3. At step 440, the morphology similarity metric MS₂ of {HS₂} is computed, and compared to a threshold value MS_{TH2}. The MS_{TH2} can be the same as MS_{TH1} in one example, or different than MS_{TH1} in another example. If MS₂ is greater than MS_{TH2}, then at step 470 the second group {HS₂} is deemed satisfactory, and can be selected as the heart sound ensemble for computing the ensemble average. If MS₂ is no greater than MS_{TH2}, then at step 450 the morphology similarity metric MS₂ of the second group is compared to the morphology similarity metric MS₁ of the first group, and whichever group that has a greater similarity metric value can be selected as the heart sound ensemble, that is. The heart sound ensemble circuit 323 may align the heart sound segments of the selected group with respect to their respective fiducial points, such as ventricular activations (e.g., R waves in an ECG), and computes an ensemble average of the heart sound segments as the representative heart sound segment.

Referring back to FIG. 2, the heart sound recognition circuit 222 may use one of a time-based tracker 224 or a spectral-based tracker 225 to recognize and track a heart sound component of interest, such as a S1, S2, S3, or S4 component. The time-based tracker 224 uses a time-based tracking algorithm to recognize a heart sound component of interest from the representative heart sound segment, such as that produced by the heart sound ensemble circuit 223. The time-based tracking algorithm involves, among other information, amplitude and timing information of the heart sound segment. An example of the time-based tracking algorithm used by the time-based tracker 224 is described in Patangay et al. U.S. Pat. No. 7,853,327, entitled “HEART SOUND TRACKING SYSTEM AND METHOD,” which refers to, among other things, recognizing a heart sound component from a heart sound waveform using a first intra heart sound energy indication and a corresponding first intra heart sound time indication using the heart sound information from the particular heart sound waveform and the heart sound information from at least one other heart sound waveform, the disclosure of which is incorporated by reference herein in its entirety. The spectral-based tracker 225 uses a spectral-based tracking algorithm to recognize a heart sound component of interest from the representative heart sound segment, such as that produced by the heart sound ensemble circuit 223. The spectral-based tracking algorithm uses, among other information, one or more spectral features that can be derived from the heart sound segment. An example of the spectral-based tracking algorithm used by the spectral-based tracker 225 is described in Goftari et al. U.S. Patent Application No. 63/432,649 entitled “ENTROPY BASED HEART SOUND TRACKING,” which describes heart sound component recognition from a heart sound waveform using a complexity measure, including an entropy (or varieties thereof), obtained from power spectra of a heart sound signal, the disclosure of which is incorporated by reference herein in its entirety. The spectral entropy-based approach quantifies complexities (or non-uniformities) of power spectral magnitudes of a heart sound signal or a portion thereof such as within a cardiac cycle. A high spectral entropy indicates more uniformly distributed signal energy across a wide frequency range in the frequency domain, while a low spectral entropy indicates less uniformity in the signal energy distribution. A wideband signal (e.g., a white noise signal), characterized by signal energy spreading almost uniformly over a wide frequency range, and therefore has large spectral entropy. Conversely, a narrowband signal has most of its signal energy focused on a narrow frequency band and less uniformly distributed, and therefore a smaller spectral entropy. Certain heart sound components, such as S1 and S2, are narrowband signals. For example, S1 power generally falls within approximately 10-50 Hz. and S2 power generally falls within approximately 20-70 Hz. As such, S1 and S2 generally have lower spectral entropy values.

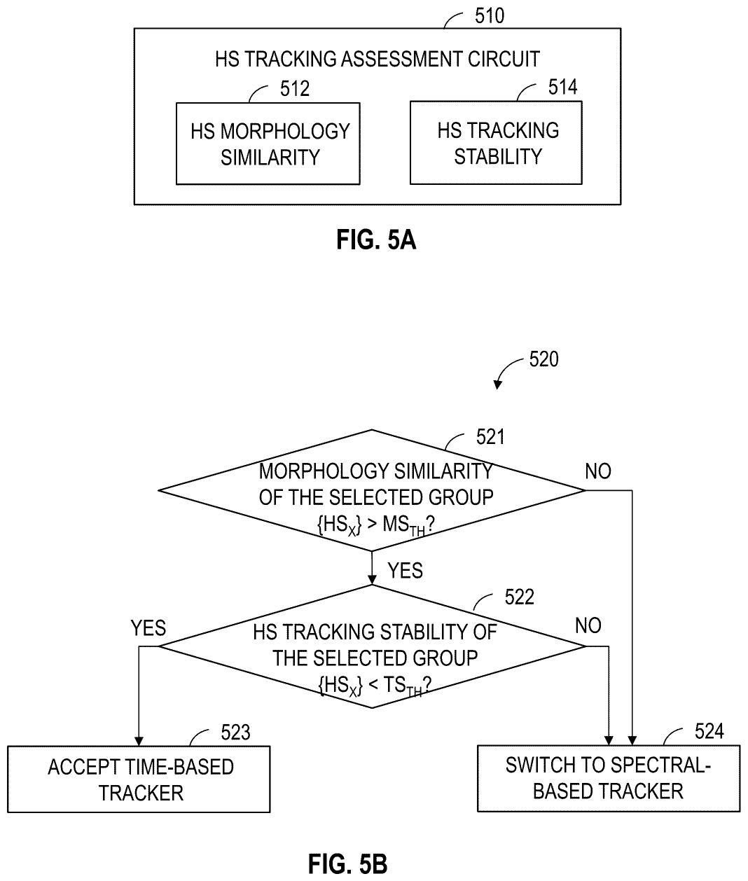

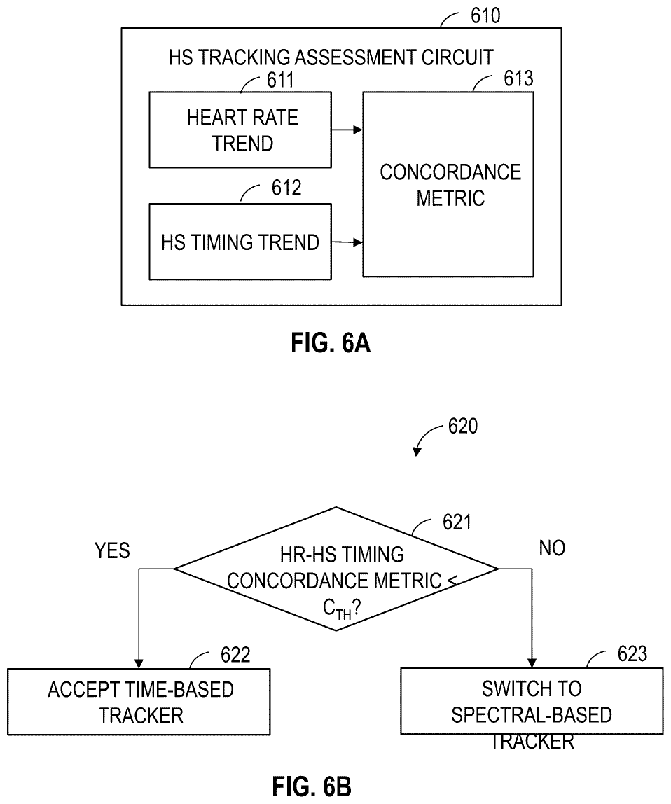

The spectral entropy-based heart sound recognition combines the advantages of frequency-domain spectral analysis and Shannon's entropy, both of which can be more robust to random noise and interferences from various sources than the time-domain, amplitude-based heart sound detection method. However, compared to time-based heart sound recognition method, the spectral entropy-based approach is generally more computationally intensive, and may require more computing resources and consume more power of the implantable device. To improve heart sound recognition performance with balanced tradeoffs between accuracy and efficiency, in an example, the heart sound recognition circuit 222 may select between the time-based tracker 224 and the spectral-based tracker 225 to recognize and track a heart sound component of interest. Alternatively, the heart sound recognition circuit 222 may perform an initial recognition of a heart sound component of interest using a computationally less intensive algorithm (such as the time-based tracking algorithm as used by the time-based tracker 224), and based on an evaluation of the performance of the initial recognition, determine whether or not to switch to a computationally more intensive algorithm (such as the spectral entropy-based tracking algorithm as used by the spectral-based tracker 225) to further recognize the heart sound component of interest. As illustrated in FIG. 2, the heart sound tracking assessment circuit 226 may evaluate the performance of one or more of the time-based heart sound tracking algorithm or the spectral-based tracking algorithm based at least on morphological or timing information of the heart sound component of interest. FIGS. 5A-5B and 6A-6B illustrate embodiments of the heart sound recognition circuit 222 to assess a quality of the time-based tracker 224 in recognizing a heart sound component of interest. FIG. 5A illustrates a heart sound tracking assessment circuit 510 configured to generate a heart sound recognition performance index using a heart sound morphology similarity metric 512 and a heart sound tracking stability 514. The heart sound morphology similarity metric 512 is a measure of similarity among at least the subset of the heart sound segments being used for generating the representative heart sound segment, such as the selected group of heart sound segments (e.g., the first group {HS₁} or the second group {HS₂}). As described above with respect to FIGS. 3-4, the morphology similarity metric may be computed using heart sound waveform portions within a specified window, such as a S2 detection window for evaluating an S2 morphology similarity metric. The morphology similarity metric may be computed as accumulated deviations or RMS values of heart sound waveforms within the same group in one example, or correlations between morphological features of the heart sound waveforms within the selected group.

The heart sound tracking stability 514 is a measure of stability of timings of heart sound component of interest (e.g., S2) over time at different heart rates. In the case of recognizing and tracking S2 heart sound, aortic stenosis (AS) is one of factors affecting S2 timing stability in heart sounds. The heart sound tracking stability 514 may be approximated by an AS index indicating a presence or severity of AS. The AS index measures a physical property of the aortic valve, such as aortic valve surface area or a change in surface area thereof. The surface area of the aortic value (S_{AV}) or a change in S_{AV} may be estimated using a heart sound-based Gorlin formula, as described in detail in Goftari et al. U.S. Patent Application No. 63/523,152 entitled “AORTIC STENOSIS SEVERITY QUANTIFICATION USING HEART SOUNDS,” the disclosure of which is incorporated by reference herein in its entirety. Specifically, the heart sound-based Gorlin formula, reproduced below, involves a heart rate (HR), a heart sound-based ejection time (ET) estimated as an interval between S1 and S2 heart sound components (T_{S1-S2}), and an S2 intensity such as S2 amplitude or peak power (∥S2∥). The surface area of the aortic value S_{AV} is in inverse proportion to each of HR, T_{S1-S2}, and a square root of S2 intensity. An increase in S2 intensity and/or a prolonged S1-S2 time interval may lead to a decrease in aortic valve surface area S_{AV}, indicating a presence or elevated severity of AS.

$${S}_{AV} ∝ 1 / HR * {T}_{S 1 - S 2} * \sqrt{‖ S 2 ‖} \qquad ( 1 )$$

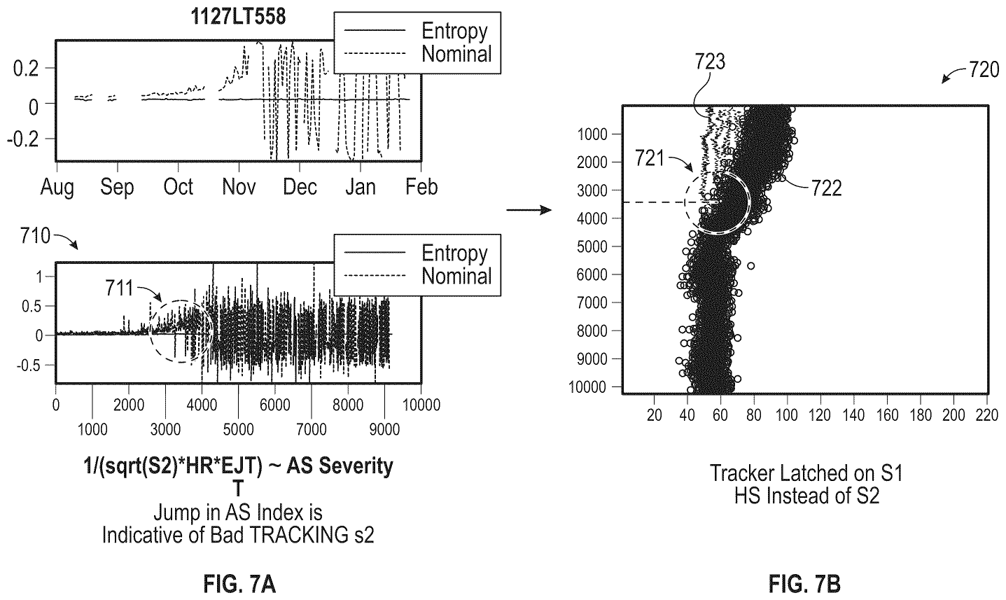

An example of quantifying heart sound tracking stability based on an aortic stenosis (AS) indication (e.g., as computed using the heart sound-based Gorlin formula), and using the same to determine whether or not to switch to a spectral-based tracker to recognize a heart sound component of interest is illustrated in FIGS. 7A-7B. FIG. 7A illustrates a time series of AS indices 710 (on the y-axis) respectively computed for consecutive heart sound segments over time (along the x-axis) using the heart sound-based Gorlin formula above. FIG. 7B illustrates a heart sound map 720 representing a plurality of heart sound segments stacked together (along the y-axis, indexed by the number of segments). Heart sound segments are extracted over multiple cardiac cycles with varying heart rates, and aligned with respect to respective fiducial points (origin of the x-axis), such as the R waves of respective cardiac cycles. Each heart sound segment (along the x-axis) has signal amplitudes color-coded or gray scale-coded to show the changes in amplitude over time. Also shown in FIG. 7B are S2 components 722 of each of the heart sound segments as recognized and tracked by the time-based tracker 224.

FIG. 7A shows a sudden “jump” 711 in AS indices at around time T (corresponding to heart sound segment indices of approximately 3400-3500), indicating an increase in heart sound tracking variability. FIG. 7B shows that, at about the same time T (corresponding to heart sound segment indices of approximately 3400-3500), S2 starts to be mis-detected at approximately timing locations of S1 components, suggesting the time-based tracker 224 latches on S1 components 723 instead of S2, thereby causing a large variability in recognized S2 locations. The example in FIGS. 7A-7B demonstrates that the S2 tracking performance (e.g., the misrecognition of S2) may be detected using the variability of AS indices (e.g., sudden jump 711) computed according to the heart sound-based Gorlin formula above,

The heart sound tracking stability 514 can be determined as a variability of AS indices (each computed as 1/HR*T_{S1-S2}*\sqrt{∥S2∥}) of the subset of the heart sound segments being used for generating the representative heart sound segment, such as the selected group of heart sound segments (e.g., the first group {HS₁} or the second group {HS₂}) produced by the heart sound ensemble circuit 223. For example, if the first group {HS₁} is selected according to method 400 as illustrated in FIG. 4, then AS indices may be computed for each of the K₁ heart sound segments of that group. Similarly, if the first group {HS₁} is selected according to method 400, then the AS indices may be computed for each of the K₂ heart sound segments of that group. The heart sound tracking stability 514 can then be determined as a variability of the AS indices of the selected group.

It should be realized that using the AS index as described above is an non-limiting example of determining the heart sound tracking stability 514. Other metrics, such as the heart sound-based ejection time (ET) T_{S1-S2}, or the S2 intensity ∥S2∥, may each be used to approximate the heart sound tracking stability 514. For example, the heart sound tracking stability 514 may be determined based on a deviation of T_{S1-S2} from an ET threshold, or based on a deviation of ∥S2∥ from an S2 intensity threshold.

FIG. 5B is a flowchart illustrating an example method 520 that may be used by the heart sound tracking assessment circuit 510 of FIG. 5A to assess the performance of the time-based tracker 224, and to determine whether or not to switch to the spectral-based tracker to recognize a heart sound component of interest (e.g., S2) using the assessed performance. At step 521, the morphology similarity of the selected group of heart sound segments (e.g., {HS₁} or {HS₂}) can be compared to a similarity threshold MS_{TH}. If the morphology similarity is above the threshold MS_{TH}, then at step 522 the heart sound tracking stability of the selected group of heart sound segments can be checked against a stability threshold TS_{TH}. If the heart sound tracking stability is below the variability threshold TS_{TH}, then at step 523, the time-based tracker 224 is deemed acceptable. If at step 521 the morphology similarity is below the threshold MS_{TH}, or if the heart sound tracking stability is greater than the variability threshold TS_{TH}, then the time-based tracker is deemed inacceptable, and at step 524, the spectral-based tracker 225 can be used to recognize and track the heart sound component of interest.

FIG. 6A illustrates a heart sound tracking assessment circuit 610 configured to generate a heart sound recognition performance index using a concordance metric 613 between a heart rate trend 611 and a heart sound timing trend 612. The heart rate trend 611 and the heart sound timing trend 612 may each be calculated using at least the subset of the heart sound segments being used for generating the representative heart sound segment, such as the selected group of heart sound segments (e.g., the first group {HS₁} or the second group {HS₂}) produced by the heart sound ensemble circuit 223. The heart rate trend 611 represents a trend of instantaneous heart rates of the heart beats corresponding to the selected group of heart sound segments. The heart sound timing trend 612 represents a trend of timings of a heart sound component of interest, such as S2, detected from the selected group of heart sound segments relative to respective fiducial points. A concordance metric 613, indicative of a degree of agreement between the change in heart rate and the change in heart sound component timing over time, can be computed using the heart rate trend 611 and the heart sound timing trend 612. In one example, the concordance metric 613 may be computed using a correlation between the heart rate trend 611 and the heart sound timing trend 612. In another example, linear or nonlinear regression analysis may be used to generate a first regression line or regression curve of heart rates and a second regression line or regression curve of heart sound component timings (e.g., S2 timings). The concordance metric 613 may be determined based on a degree of deviation between the first and the second regression lines or regression curves.

FIG. 6B is a flowchart illustrating an example method 620 that may be used by the heart sound tracking assessment circuit 610 of FIG. 6A to assess the performance of the time-based tracker 224, and to determine whether or not to switch to the spectral-based tracker to recognize a heart sound component of interest (e.g., S2) using the assessed performance. At step 621, a concordance metric may be computed using the heart rate trend 611 and the heart sound timing trend 612. The concordance metric may be compared to checked against a concordance threshold C_{TH}. If the concordance metric is below the concordance threshold C_{TH}, then at step 622, the time-based tracker 224 is deemed acceptable. If at step 621 the concordance metric is above the threshold C_{TH}, then the time-based tracker 224 is deemed inacceptable, and at step 623 the spectral-based tracker 225 is used to recognize and track the heart sound component of interest.

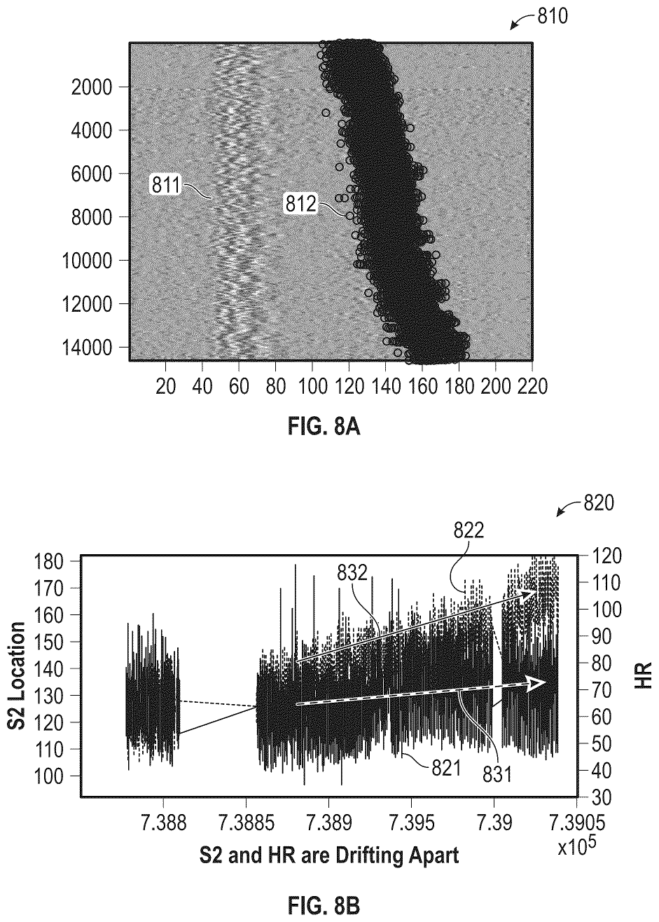

An example of determining a concordance between heart rates and S2 timings, and using the same to determine whether or not to switch to a spectral-based tracker to recognize a heart sound component of interest is illustrated in FIGS. 8A-8B. FIG. 8A illustrates a heart sound map 810 representing a plurality of heart sound segments stacked together (along the y-axis, indexed by the number of segments). Each heart sound segment (on the x-axis) has the signal amplitudes color-coded or gray scale-coded to show the changes in amplitude over time. Also shown in FIG. 8A are S1 components 811 and S2 components 812 of each of the heart sound segments. S2 may be recognized and tracked using the time-based tracker 224, and S2 timings may be determined relative to respective fiducial points, such as the R wave of respective cardiac cycles. FIG. 8B illustrates instantaneous heart rates 821 of heart beats corresponding to the heart sound segments as shown in FIG. 8A, and S2 timings 822 (with respect to respective fiducial points) determined by the detected S2 components 812. Regression analysis can be performed to determine a heart rate regression line 831 from the instantaneous heart rates 821, and a S2 timing regression line 832 from the S2 timings 822. FIG. 8B shows that the heart rate trend and the S2 timing trend are drifting apart, which may be quantified as a difference between the respective slopes of the heart rate regression line 831 and the S2 timing regression line 832. If the difference exceeds a threshold, then the time-based tracker 224 is deemed inacceptable, and a decision or a recommendation can be made to the user to switch to the spectral-based tracker 225 to recognize and track S2 component.

Referring back to FIG. 2, the physiologic event detector 227 can detect a physiologic event using one or more of the heart sound components detected by the heart sound recognition circuit 222. In some examples, the physiologic event detector 227 may generate a heart sound metric using the detected heart sound components, and detect the physiologic event using the heart sound metrics, optionally along with other physiologic information acquired from the patient. Examples of the heart sound metric may include an intensity (e.g., amplitude or signal energy under the curve) of a heart sound component, or one or more heart sound-based cardiac timing intervals, such as a pre-ejection period (PEP) such as measured between the onset of the QRS to the S1 heart sound, a systolic timing interval (STI) such as measured between the onset of the QRS complex on the ECG to the S2 heart sound, a left-ventricular ejection time (LVET) such as measured as an interval between S1 and S2 heart sounds, or a diastolic timing interval (DTI) such as measured between the S2 heart sound and the onset of the subsequent QRS complex on the ECG, among others. These heart sound-based cardiac timing intervals may be correlated with cardiac contractility or cardiac diastolic function of the heart. The heart sound metrics may further include PEP/LVET ratio, STI/DTI ratio, STI/cycle length (CL) ratio, or DTI/CL ratio, or other composite metrics.

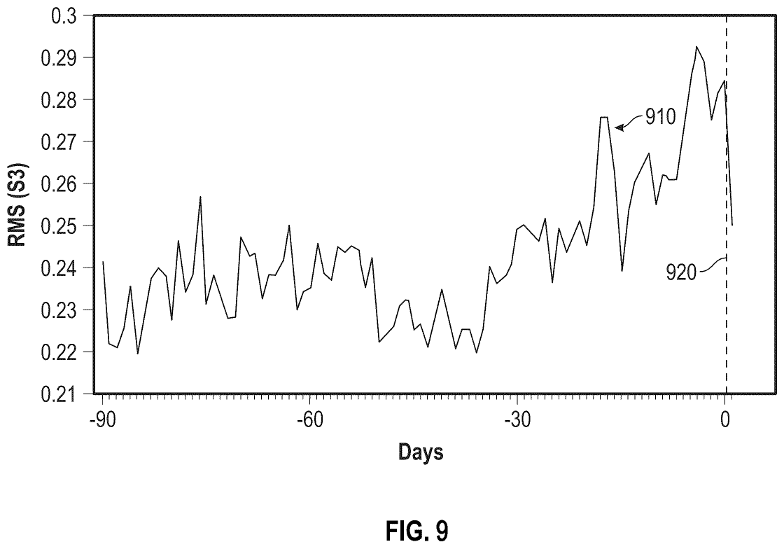

In an example, the physiologic event detector 227 may track a heart sound component over time and generate a heart sound trend, such as an S1 amplitude trend, an S2 amplitude trend, an S1 timing trend, an S2 timing trend, or a trend of heart sound-based cardiac timing intervals (e.g., a PEP trend, an STI trend, an LVET trend, a DEI trend, etc.). Based on the heart sound trend, the physiologic event detector 227 can generate a cardiac function indicator indicating myocardial contractility, cardiac synchrony, and cardiac hemodynamics. In an example, the physiologic event detector 227 may detect a cardiac arrhythmia episode, or distinguish between different arrhythmias (e.g., atrial tachyarrhythmia, supraventricular tachyarrhythmia, or ventricular tachyarrhythmia) using one or more of S1 intensity, S2 intensity, or a measure of STI. For example, a reduction in S1 intensity may be indicative of reduced cardiac contractility, and a reduction in S2 intensity may be indicative of reduced cardiac output, both of which may be used to detect cardiac arrhythmias and deterioration of cardiac hemodynamics during arrhythmia. In another example, the heart sound metrics, such as S3 intensity, may be used to detect WHF. An increase in S3 intensity indicates reduced ventricular compliance and deteriorating diastolic function, signifying occurrence of WHF. Additionally or alternatively, a reduction in S1 intensity, or a reduction in STI may indicate poor cardiac contractility or reduced electromechanical coupling, which signifies occurrence of WHF. An example of detecting WHF based on heart sound components recognized and tracked in accordance with various examples described in this document, such as the time-based tracking algorithm and/or the spectral-based tracking algorithms as described above with respect to FIGS. 2, 5A-5B, and 6A-6B, is described further below with reference to FIG. 9. The physiologic event detector 227 may additionally or alternatively detect respiratory, renal, neurological, among other medical conditions, based on the heart sound metrics.

The user interface 230 may include an input unit and an output unit. In an example, at least a portion of the user interface 230 may be implemented in the external system 105. The input unit may receive user input for programming the data receiver circuit 210 and the controller circuit 220, such as parameters for sensing the heart sound signal, generating a heart sound ensemble, detecting a heart sound component of interest using one or more of the time-based tracker or the spectral-based tracker, or detecting a physiologic event, among others. The input unit may include a keyboard, on-screen keyboard, mouse, trackball, touchpad, touchscreen, or other pointing or navigating devices. The output unit may include a display for displaying the sensed heart sound signal, the representative heart sound segments, the spectral entropy time series, the heart sound metrics, information about the detected physiologic events, and any intermediate measurements or computations, among others. The output unit may also present to a user, such as via a display unit, the therapy titration protocol and recommended therapy, including a change of parameters in the therapy provided by an implanted device, the prescription to get a device implanted, the initiation or change in a drug therapy, or other treatment options of a patient. The output unit may include a printer for printing hard copies of information that may be displayed on a display unit. The signals and information may be presented in a table, a chart, a diagram, or any other types of textual, tabular, or graphical presentation formats. The presentation of the output information may include audio or other media format. In an example, the output unit may generate alerts, alarms, emergency calls, or other forms of warnings to signal the system user about the detected medical events.

The therapy circuit 240 may be configured to deliver a therapy to the patient, such as in response to the detected physiologic event. The therapy may be preventive or therapeutic in nature such as to modify, restore, or improve patient neural, cardiac, or respiratory functions. Examples of the therapy may include electrostimulation therapy delivered to the heart, a nerve tissue, other target tissues, a cardioversion therapy, a defibrillation therapy, or drug therapy including delivering drug to the patient. In some examples, the therapy circuit 240 may modify an existing therapy, such as adjust a stimulation parameter or drug dosage.

FIG. 9 illustrates generally an example of detecting worsening heart failure (WHF) based at least in part on heart sound components (e.g., S2 and S3) recognized and tracked in accordance with various examples described in this document, such as the time-based tracking algorithm and/or the spectral-based tracking algorithms as described above with respect to FIGS. 2, 5A-5B, and 6A-6B. From each cardiac cycle, S3 can be detected based the timing of S2 detected within that cardiac cycle using the entropy-based approach as described above. In an example, S3 can be detected within a S3 detection window that begins at the S2 timing, or a specified offset following the detected S2 (e.g., approximately 50-125 msec following the S2 timing) and lasts a duration of approximately 125 msec. An S3 metric, such as a root-mean-square (RMS) of S3, can be computed and tracked over time, as represented by a daily S3 trend 910. An WHF detection index can be computed using S3 RMS value, optionally along with other sensor information, through a weighted combination unit or other types of sensor fusion engine. In this example, daily S3 is tracked for approximately 90 days until the WHF detection index value reaches and exceeds a predetermined WHF detection threshold, at which point (time zero 920) an WHF event is deemed detected, and a user can be alerted. In an example, the WHF detection index may include an intensity (e.g., amplitude or RMS value) ratio of S3 to S1 components. Pramodsingh et al. U.S. patent application Ser. No. 15/473,783 entitled “SYSTEMS AND METHODS FOR DETECTING WORSENING HEART FAILURE” describes techniques for detecting worsening cardiac events such as WHF events using information from multiple sensors including heart sounds, the disclosure of which is incorporated by reference herein in its entirety.

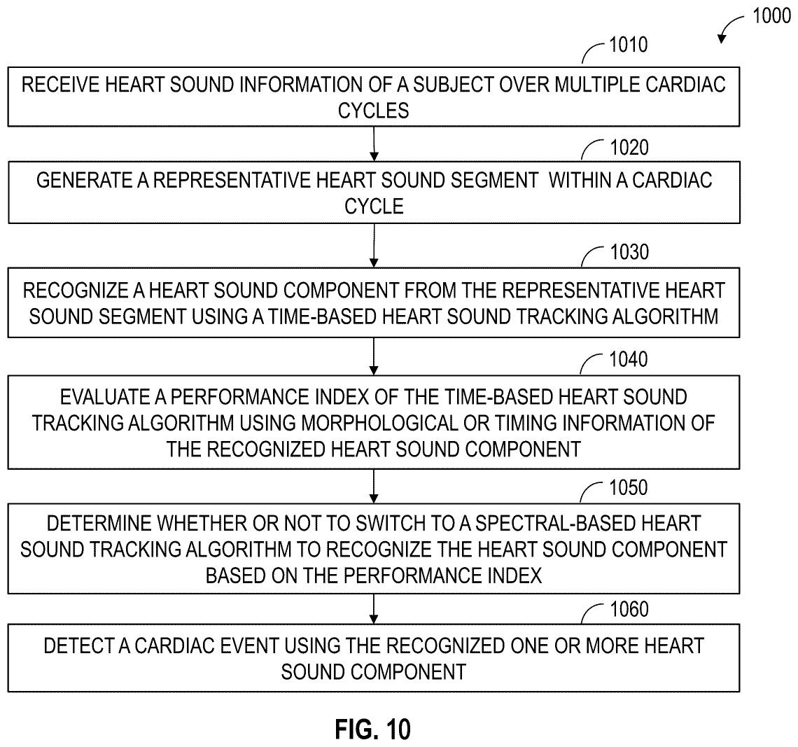

FIG. 10 illustrates generally an example of a method 1000 for recognizing and tracking a heart sound component from a heart sound signal and using the recognized heart sound component to detect a physiologic event. The method 1000 may be implemented and executed in an ambulatory medical device such as an implantable or wearable medical device, or in a remote patient management system. In an example, the method 800 may be implemented in and executed by the IMD 102 or the WMD 103, the external system 105, or the heart sound-based physiologic event detection system 200.

The method 1000 begins at step 1010 to receive physiologic information of a subject including heart sounds over multiple cardiac cycles. The heart sounds may be detected using a sensor associated with or included in an ambulatory or wearable device. In some examples, endocardial acceleration signals sensed from inside the heart may be used to analyze heart sounds. Other physiologic information may also be received, including a cardiac electrical signal such as an electrocardiography (ECG) or an intracardiac electrogram (EGM), heart rate, signals indicative of cardiac mechanical activity including, for example, thoracic or cardiac impedance signal, arterial pressure signal, pulmonary artery pressure signal, left atrial pressure signal, RV pressure signal, LV pressure signal, heart sounds or endocardial acceleration signal, physiologic response to activity, apnea hypopnea index, one or more respiration signals such as a respiration rate signal or a tidal volume signal, among others.

At step 1020, a representative heart sound segment may be generated using at least a portion of the received heart sounds. An example of the representative heart sound segment is an ensemble average of a plurality of heart sound segments taken from respective cardiac cycles, and time-aligned with respect to respective fiducial points, such as R waves in the multiple cardiac cycles of an ECG signal. In some examples, a subset of the heart sound segments that satisfy a specified condition may be selected and used to establish the representative heart sound segment. The selection criteria may include one or more of heart rate, heart rate variability, activity level, respiration rate, or time of a day, among others. In one example, the subset of the heart sound segments may be selected to correspond to heart rates falling below a predetermined or user-set heart rate threshold (e.g., 100-120 bpm). In another example, the subset of the heart sound segments may be selected to correspond to heart rates or cycle lengths falling within a specified range, such as substantially the same cycle length within a margin of +/−100 milliseconds, or substantially the same heart rate within a specific heart rate variability margin of +/−5 beats per minute (bpm) in one example, or +/−10 bpm in another example.

In some examples, the heart sound segments over multiple cardiac cycles may be sorted according to a specific order of the instantaneous heart rates of heart beats corresponding to the heart sound segments. From the sorted plurality of heart sound segments, at least a first group of heart sound segments corresponding to first heart beats, denoted by {HS_{HR1}}, and a second group of heart sound segments corresponding to second heart beats having different heart rates than the first heart beats, denoted by {HS_{HR2}}, may be identified. Morphology similarities among the heart sound segments may be evaluated respectively for each of the groups, including, for example, a first morphology similarity metric among the first group of heart sound segments {HS_{HR1}}, and a second morphology similarity metric among the second group of heart sound segments {HS_{HR2}}. Selection can be made between the first group {HS_{HR1}} and the second group{HS_{HR2}}, based on one or more of the first morphology similarity metric or the second morphology similarity metric, such as in accordance with the method as described above with respect to FIGS. 3 and 4.

At step 1030, a heart sound component of interest (e.g., S2) may be recognized from the representative heart sound segment using a time-based heart sound tracking algorithm, such as implemented in the time-based tracker 224. The time-based tracking algorithm uses, among other information, amplitude and timing information of the heart sound segment. An example of the time-based tracking algorithm to be used by is described in Patangay et al. U.S. Pat. No. 7,853,327, entitled “HEART SOUND TRACKING SYSTEM AND METHOD,” which describes recognizing a heart sound component from a heart sound waveform using a first intra heart sound energy indication and a corresponding first intra heart sound time indication using the heart sound information from the particular heart sound waveform and the heart sound information from at least one other heart sound waveform.

At step 1040, a performance index of the time-based heart sound tracking algorithm may be evaluated, such as using the heart sound tracking assessment circuit 226. The evaluation may be based on morphological or timing information of the recognized heart sound component, as described above with respect to FIGS. 5A-5B and 6A-6B. One method, as described above with respect to FIGS. 5A-5B, involves the use of a heart sound morphology similarity metric and a heart sound tracking stability. The heart sound morphology similarity metric is a measure of the similarity among at least the subset of the heart sound segments being used for generating the representative heart sound segment, such as the selected group of heart sound segments (e.g., the first group {HS₁} or the second group {HS₂}). The heart sound tracking stability is a measure of stability of timings of heart sound component of interest (e.g., S2) over time at different heart rates. In an example of recognizing S2 heart sound, the heart sound tracking stability may be quantified using a variability of aortic stenosis (AS) index, which can be computed using the heart sound-based Gorlin formula as described above.

Another method of assessing the performance of the time-based heart sound tracking algorithm is described above with respect to FIGS. 6A-6B, which involves a determination of a concordance metric between a heart rate trend and a heart sound timing trend. The heart rate trend represents a trend of instantaneous heart rates of the heart beats corresponding to the selected group of heart sound segments. The heart sound timing trend represents a trend of timings of a heart sound component of interest, such as S2, detected from the selected group of heart sound segments relative to respective fiducial points. The heart rate trend and the heart sound timing trend may each be calculated using at least the subset of the heart sound segments being used for generating the representative heart sound segment, such as the selected group of heart sound segments (e.g., the first group {HS₁} or the second group {HS₂}). The concordance metric is indicative of a degree of agreement between the change in heart rate and the change in heart sound component timing over time. In an example, the concordance metric may be computed using a correlation between the heart rate trend and the heart sound timing trend. In another example, linear or nonlinear regression analysis may be used to generate a first regression line or regression curve of heart rates and a second regression line or regression curve of heart sound component timings (e.g., S2 timings), and the concordance metric may be determined based on a degree of deviation between the first and the second regression lines or regression curves.

At step 1050, based on the evaluated performance index, a decision or a recommendation to the user can be made whether or not to switch to a spectral-based heart sound tracking algorithm to recognize the heart sound component from the representative heart sound segment. An example method of using the heart sound morphology similarity metric and the heart sound tracking stability to assess the performance of the time-based S2 recognition algorithm and to determine whether or not to switch to the spectral-based tracker to recognize the S2 is described with respect to FIG. 5B. An example method of using the concordance between the heart rate trend and the heart sound component timing (e.g., S2 timing) trend to assess the performance of the time-based S2 recognition algorithm and to determine whether or not to switch to the spectral-based tracker to recognize S2 heart sound is described with respect to FIG. 6B.

At step 1060, a cardiac event may be detected using the one or more heart sound components, such as using the physiologic event detector 227. The physiologic event may include an indicator of myocardial contractility, cardiac synchrony, and cardiac hemodynamics, a cardiac arrhythmia episode, or a worsening heart failure (WHF) event. An example of detecting a WHF event based at least in part on heart sound components (e.g., S2 and S3 components) recognized and tracked in accordance with various examples described in this document is described above with respect to FIG. 9. In some examples, the heart sound metric may be used together with other sensor information to detect respiratory, renal, neurological, among other medical conditions, based on the heart sound metrics generated from the heart sound segment. In some examples, based on the detection of the physiologic event, a recommendation may be generated and provided to the user. The recommendation may include one or more of further diagnostic tests to be performed, adjustment of one or more parameters for detecting the physiologic event, or therapy to be delivered or adjustment of one or more therapy parameters. The system user may review and adjudicate the detected physiologic event, and reprogram one or more detection or therapy parameters, such as using the user interface 230. In some examples, therapy may be delivered to the patient in response to the detected physiologic event, such as via the therapy circuit 240 as illustrated in FIG. 2. Examples of the therapy may include electrostimulation therapy delivered to the heart, a nerve tissue, other target tissues, a cardioversion therapy, a defibrillation therapy, or drug therapy including delivering drug to a tissue or organ. In some examples, an existing therapy or treatment plan may be modified to treat the detected arrhythmia, such as modify patient follow-up schedule, or adjust a stimulation parameter or drug dosage.

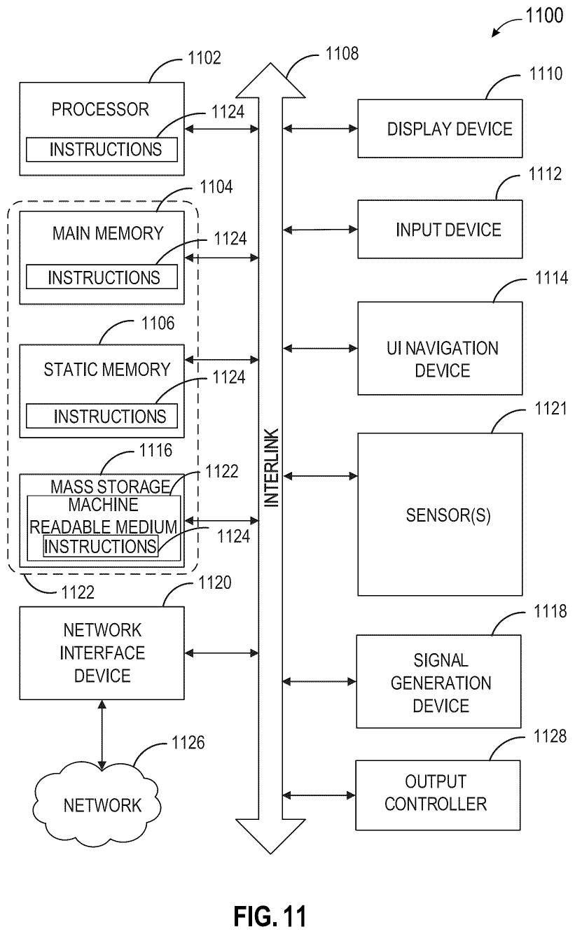

FIG. 11 illustrates generally a block diagram of an example machine 1100 upon which any one or more of the techniques (e.g., methodologies) discussed herein may perform. Portions of this description may apply to the computing framework of various portions of the IMD 102, the WMD 103, the external system 105, or the heart sound-based physiologic event detection system 200.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1100 may include a hardware processor 1102 (e.g., a central processing unit (CPU), a graphics processing unit (GPU), a hardware processor core, or any combination thereof), a main memory 1104 and a static memory 1106, some or all of which may communicate with each other via an interlink (e.g., bus) 1108. The machine 1100 may further include a display unit 1110 (e.g., a raster display, vector display, holographic display, etc.), an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device (e.g., drive unit) 1116, a signal generation device 1118 (e.g., a speaker), a network interface device 1120, and one or more sensors 1121, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1116 may include a machine readable medium 1122 on which is stored one or more sets of data structures or instructions 1124 (e.g., software) embodying or utilized by any one or more of the techniques or functions described herein. The instructions 1124 may also reside, completely or at least partially, within the main memory 1104, within static memory 1106, or within the hardware processor 1102 during execution thereof by the machine 1100. In an example, one or any combination of the hardware processor 1102, the main memory 1104, the static memory 1106, or the storage device 1116 may constitute machine readable media.

While the machine-readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1124 may further be transmitted or received over a communication network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communication network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should therefore be determined with references to the appended claims, along with the full scope of equivalents to which such claims are entitled.

What is claimed is:

1. A medical-device system, comprising:
a data receiver circuit configured to receive heart sound information over multiple cardiac cycles; and
a heart sound recognition circuit configured to:
generate a representative heart sound segment within a cardiac cycle using at least a portion of the received heart sound information;
recognize a heart sound component from the representative heart sound segment using a time-based heart sound tracking algorithm;
evaluate a performance index of the time-based heart sound tracking algorithm using morphological or timing information of the recognized heart sound component; and
based on the evaluated performance index, determine whether or not to switch to a spectral-based heart sound tracking algorithm to recognize the heart sound component from the representative heart sound segment.

2. The medical-device system of claim 1, wherein:
the time-based heart sound tracking algorithm recognizes the heart sound component using amplitude and timing information of the representative heart sound segment; and
the spectral-based heart sound tracking algorithm recognizes the heart sound component using one or more spectral entropy values calculated from the representative heart sound segment.

3. The medical-device system of claim 1, wherein the portion of the received heart sound information includes a plurality of heart sound segments corresponding to respective heart beats,
wherein the heart sound recognition circuit is configured to generate the representative heart sound segment using an ensemble average of at least a subset of the plurality of heart sound segments.

4. The medical-device system of claim 3, wherein the data receiver circuit is configured to receive information about instantaneous heart rates of the heart beats that correspond to the plurality of heart sound segments,
wherein the at least the subset of the plurality of heart sound segments being used for generating the representative heart sound segment corresponds to heart beats falling within a pre-determined heart rate range.

5. The medical-device system of claim 4, wherein to generate the representative heart sound segment, the heart sound recognition circuit is configured to:
sort the plurality of heart sound segments according to a specific order of the instantaneous heart rates of the heart beats corresponding to the plurality of heart sound segments;
identify, from the sorted plurality of heart sound segments, at least a first group of heart sound segments corresponding to first heart beats, and a second group of heart sound segments corresponding to second heart beats having different heart rates than the first heart beats;
determine a first morphology similarity metric of the first group of heart sound segments, and a second morphology similarity metric of the second group of heart sound segments;
select a group between the first group and the second group based on one or more of the first morphology similarity metric or the second morphology similarity metric; and
generate the representative heart sound segment using an ensemble average of the selected group of heart sound segments.

6. The medical-device system of claim 5, wherein the first group and the second group each have at least a specific minimum number of heart sound segments.

7. The medical-device system of claim 5, wherein the first heart beats corresponding to the first group of heart sound segments and the second heart beats corresponding to the second group of heart sound segments each have respective instantaneous heart rates falling below a heart rate threshold and satisfying a heart rate variability or range requirement.

8. The medical-device system of claim 5, wherein the plurality of heart sound segments are sorted according to an ascending order of the instantaneous heart rates of the heart beats,
wherein the second heart beats corresponding to the second group of heart sound segments have higher heart rates than the first heart beats corresponding to the first group of heart sound segments,
wherein to select between the first group and the second group includes:
to select the first group if (i) the first morphology similarity metric is above a first threshold, or (ii) both the first and the second morphology similarity metrics are below respective thresholds and the first morphology similarity metric is greater than the second morphology similarity metric; and
to select the second group if (i) the first morphology similarity metric is below the first threshold and the second morphology similarity metric is above a second threshold, or (ii) both the first and the second morphology similarity metrics are below the respective thresholds and the second morphology similarity metric is greater than the first morphology similarity metric.

9. The medical-device system of claim 3, wherein the heart sound recognition circuit is configured to evaluate the performance index of the time-based heart sound tracking algorithm using a morphology similarity metric among the at least the subset of the plurality of heart sound segments being used for generating the representative heart sound segment.

10. The medical-device system of claim 9,
wherein to evaluate the performance index of the time-based heart sound tracking algorithm includes further using a heart sound tracking stability indicative of variability of timings of the heart sound component respectively determined from the at least the subset of the plurality of heart sound segments being used for generating the representative heart sound segment,
wherein to determine whether or not to switch to the spectral-based heart sound tracking algorithm, the heart sound recognition circuit is configured to:
determine not to switch to the spectral-based heart sound tracking algorithm to recognize the heart sound component from the representative heart sound segment if (i) the morphology similarity metric exceeds a similarity threshold, and (ii) the heart sound tracking stability is below a variability threshold; and
determine to switch to the spectral-based heart sound tracking algorithm to recognize the heart sound component from the representative heart sound segment if (i) the morphology similarity metric is no greater than the similarity threshold, or (ii) the heart sound tracking stability exceeds the variability threshold.

11. The medical-device system of claim 10, wherein the heart sound tracking stability is indicative of variability of timings of S2 component,
wherein the heart sound recognition circuit is configured to determine aortic stenosis (AS) indices indicating a presence or severity of AS for each of the at least the subset of the plurality of heart sound segments being used for generating the representative heart sound segment, and to determine the heart sound tracking stability using a variability of the determined AS indices.

12. The medical-device system of claim 3,
wherein to evaluate the performance index of the time-based heart sound tracking algorithm, the heart sound recognition circuit is configured to:
generate a heart sound timing trend comprising a time series of timings of the heart sound component identified from the at least the subset of the plurality of heart sound segments using the time-based heart sound tracking algorithm;
generate a heart rate trend comprising a time series of instantaneous heart rates of the heart beats corresponding to the at least the subset of the plurality of heart sound segments; and
evaluate the performance index of the time-based heart sound tracking algorithm using a concordance metric between the heart sound timing trend and the heart rate trend,
wherein to determine whether or not to switch to the spectral-based heart sound tracking algorithm, the heart sound recognition circuit is configured to:
determine not to switch to the spectral-based heart sound tracking algorithm to recognize the heart sound component from the representative heart sound segment if the concordance metric falls below a threshold; and
determine to switch to the spectral-based heart sound tracking algorithm to recognize the heart sound component from the representative heart sound segment if the concordance metric exceeds the threshold.

13. The medical-device system of claim 1, further comprising a physiological event detector configured to a detect a cardiac event using the recognized heart sound component.

14. A method of detecting a cardiac event, comprising:
receiving heart sound information over multiple cardiac cycles;
generating a representative heart sound segment within a cardiac cycle using at least a portion of the received heart sound information;
recognizing a heart sound component from the representative heart sound segment using a time-based heart sound tracking algorithm;
evaluating a performance index of the time-based heart sound tracking algorithm using morphological or timing information of the recognized heart sound component;
based on the evaluated performance index, determining whether or not to switch to a spectral-based heart sound tracking algorithm to recognize the heart sound component from the representative heart sound segment; and
detecting the cardiac event using the recognized heart sound component.

15. The method of claim 14,
wherein the time-based heart sound tracking algorithm recognizes the heart sound component using amplitude and timing information of the representative heart sound segment,
wherein the spectral-based heart sound tracking algorithm recognizes the heart sound component using one or more spectral entropy values calculated from the representative heart sound segment.

16. The method of claim 14, wherein the portion of the received heart sound information includes a plurality of heart sound segments corresponding to respective heart beats, wherein generating the representative heart sound segment includes:
sorting the plurality of heart sound segments according to a specific order of instantaneous heart rates of the heart beats corresponding to a plurality of heart sound segments;
identifying, from the sorted plurality of heart sound segments, at least a first group of heart sound segments corresponding to first heart beats, and a second group of heart sound segments corresponding to second heart beats having different heart rates than the first heart beats;
determining a first morphology similarity metric of the first group of heart sound segments, and a second morphology similarity metric of the second group of heart sound segments;
selecting a group between the first group and the second group based on one or more of the first morphology similarity metric or the second morphology similarity metric; and
generating the representative heart sound segment using an ensemble average of the selected group of heart sound segments.

17. The method of claim 16,
wherein the first group and the second group each have at least a specific minimum number of heart sound segments,
wherein the first heart beats corresponding to the first group of heart sound segments and the second heart beats corresponding to the second group of heart sound segments each have respective instantaneous heart rates falling below a heart rate threshold and satisfying a heart rate variability or range requirement.

18. The method of claim 16, wherein the plurality of heart sound segments are sorted according to an ascending order of the instantaneous heart rates of the heart beats,
wherein the second heart beats corresponding to the second group of heart sound segments have higher heart rates than the first heart beats corresponding to the first group of heart sound segments,
wherein selecting between the first group and the second group includes:
selecting the first group if (i) the first morphology similarity metric is above a first threshold, or (ii) both the first and the second morphology similarity metrics are below respective thresholds and the first morphology similarity metric is greater than the second morphology similarity metric; and
selecting the second group if (i) the first morphology similarity metric is below the first threshold and the second morphology similarity metric is above a second threshold, or (ii) both the first and the second morphology similarity metrics are below the respective thresholds and the second morphology similarity metric is greater than the first morphology similarity metric.

19. The method of claim 14, wherein the portion of the received heart sound information includes a plurality of heart sound segments corresponding to respective heart beats,
wherein evaluating the performance index of the time-based heart sound tracking algorithm includes using (i) a morphology similarity metric among at least a subset of the plurality of heart sound segments being used for generating the representative heart sound segment and (ii) a heart sound tracking stability indicative of variability of timings of a heart sound component respectively determined from the at least the subset of the plurality of heart sound segments being used for generating the representative heart sound segment,
wherein determining whether or not to switch to the spectral-based heart sound tracking algorithm includes:
determining not to switch to the spectral-based heart sound tracking algorithm to recognize the heart sound component from the representative heart sound segment if (i) the morphology similarity metric exceeds a similarity threshold, and (ii) the heart sound tracking stability is below a variability threshold; and
determining to switch to the spectral-based heart sound tracking algorithm to recognize the heart sound component from the representative heart sound segment if (i) the morphology similarity metric is no greater than the similarity threshold, or (ii) the heart sound tracking stability exceeds the variability threshold.

20. The method of claim 14, wherein the portion of the received heart sound information includes a plurality of heart sound segments corresponding to respective heart beats,
wherein evaluating the performance index of the time-based heart sound tracking algorithm includes:
generating a heart sound timing trend comprising a time series of timings of the heart sound component identified from at least a subset of the plurality of heart sound segments using the time-based heart sound tracking algorithm;
generating a heart rate trend comprising a time series of instantaneous heart rates of the heart beats corresponding to the at least the subset of the plurality of heart sound segments; and
evaluating the performance index of the time-based heart sound tracking algorithm using a concordance metric between the heart sound timing trend and the heart rate trend,
wherein determining whether or not to switch to the spectral-based heart sound tracking algorithm includes:
determining not to switch to the spectral-based heart sound tracking algorithm to recognize the heart sound component from the representative heart sound segment if the concordance metric falls below a threshold; and
determining to switch to the spectral-based heart sound tracking algorithm to recognize the heart sound component from the representative heart sound segment if the concordance metric exceeds the threshold.
